(19) United States
(12) Patent Application Publication
(10) Pub. No.: US 2016/0324505 A1
(43) Pub. Date: Nov. 10, 2016

(54) ULTRASONIC DIAGNOSTIC DEVICE

(71) Applicant: Hitachi, Ltd., Tokyo (JP)

(72) Inventors: Toshinori MAEDA, Tokyo (JP); Masaru MURASHITA, Tokyo (JP); Noriyoshi MATSUSHITA, Tokyo (JP); Yuko NAGASE, Tokyo (JP)

(73) Assignee: HITACHI, LTD., Tokyo (JP)

(21) Appl. No.: 15/038,841

(22) PCT Filed: Nov. 13, 2014
(86) PCT No.: PCT/JP2014/080702

§ 371 Date: May 24, 2016

(30) Foreign Application Priority Data
Nov. 26, 2013 (JP) 2013-243475

(51) Int. Cl. A61B 8/08; G06T 7/00; A61B 8/00

(52) U.S. Cl. CPC A61B 8/5215; A61B 8/5207; A61B 8/461; A61B 8/4444; G06T 7/0012; G06T 2207/20192; G06T 2207/10132

(57) ABSTRACT

An image processing unit (20) performs resolution conversion processing on an ultrasound image obtained on the basis of a reception signal, to generate a plurality of resolution images having mutually different resolutions. Furthermore, the image processing unit (20) performs non-linear processing on a difference image obtained by comparing the plurality of resolution images with each other, to generate boundary components related to boundaries included in the image. Moreover, a boundary-enhanced image is generated by performing enhancement processing on the ultrasound image on the basis of the generated boundary components.

### TECHNICAL FIELD

The present invention relates to an ultrasound diagnostic device, and more particularly to image processing of an ultrasound image.

### BACKGROUND ART

Techniques for enhancing a boundary of a tissue, for example, in an ultrasound image obtained by transmitting and receiving ultrasound waves are known (see Patent Documents 1 and 2).

Tone curve modification and unsharp masking are typical examples of specific boundary enhancement techniques that are conventionally known. With these techniques, however, not only the boundaries for which enhancement is desired but also other parts for which enhancement is not necessary, such as noise, may be enhanced, and also parts having a sufficient contrast may be enhanced to thereby have excessively increased contrast.

Patent Document 3 describes a method for improving the image quality of an ultrasound image by multiresolution decomposition with respect to the image.

### CITATION LIST

### Patent Literature

Patent Document 1: JP 3816151 B

Patent Document 2: JP 2012-95806 A

Patent Document 3: JP 4789854 B

### SUMMARY OF THE INVENTION

### Technical Problem

In view of the background art described above, the inventors of the present application have repeatedly conducted research and development of a technique of enhancing boundaries within an ultrasound image, and have paid particular attention to image processing to which multiple resolution decomposition is applied.

The present invention was made in the process of the research and development, and is aimed at providing a technique of enhancing a boundary within an ultrasound image using multiresolution decomposition.

### Solution to Problem

To achieve the above-described aim, in accordance with one preferred aspect, an ultrasound diagnostic device comprises a probe configured to transmit and receive ultrasound; a transmitter/receiver unit configured to control the probe to obtain a received signal of ultrasound; a resolution processing unit configured to perform resolution conversion processing with respect to an ultrasound image obtained based on the received signal, to thereby generate a plurality of resolution images having different resolutions; and a boundary component generation unit configured to generate a boundary component related to a boundary included in an image by non-linear processing applied to a differential image obtained by comparing the plurality of resolution images, wherein a boundary-enhanced image is generated by applying enhancement processing to the ultrasound image based on the boundary component which is obtained.

In a preferable specific example, the boundary component generation unit performs non-linear processing with different properties for a positive pixel value of the differential image and for a negative pixel value of the differential image.

In a preferable specific example, the boundary component generation unit performs non-linear processing such that a pixel value of the differential image having a greater absolute value is suppressed by a greater amount before being output.

In a preferable specific example, the boundary component generation unit applies, to the differential image having been subjected to the non-linear processing, weighting processing in accordance with a pixel value of the resolution image which has been used for comparison for obtaining the differential image, thereby generating the boundary component image.

In a preferable specific example, the resolution processing unit forms a plurality of resolution images having a plurality of resolutions which differ from each other stepwise, and the boundary component generation unit obtains one boundary component based on two resolution images having resolutions which differ from each other by only one step, thereby generating a plurality of boundary components corresponding to a plurality of steps, and the ultrasound diagnostic device further comprises a summed component generation unit configured to generate a summed component of an image based on a plurality of boundary components corresponding to a plurality of steps; and a summation processing unit configured to add the summed component which is generated to the ultrasound image, to thereby generate the boundary-enhanced image.

In a preferable specific example, the boundary component generation unit generates one differential image based on two resolution images having resolutions which differ from each other by only one step, and applies non-linear processing to a plurality of differential images corresponding to a plurality of steps to generate a plurality of boundary components.

### Advantageous Effects of Invention

The present invention provides a technique for enhancing a boundary within an ultrasound image using multiresolution decomposition. In accordance with a preferred aspect of the invention, the visibility of boundaries of a tissue can be increased without impairing information inherent in an ultrasound image.

### BRIEF DESCRIPTION OF DRAWINGS

FIG. 1 is a diagram illustrating an overall structure of an ultrasound diagnostic device which is suitable for implementation of the present invention.

FIG. 2 is a diagram illustrating a specific example of multiresolution decomposition.

FIG. 3 is a diagram illustrating a specific example of upsampling processing applied to a resolution image.

FIG. 4 is a diagram for explaining a differential image.

FIG. 5 is a diagram illustrating a specific example of a differential image concerning a cardiac muscle portion.

FIG. 6 is a diagram for explaining summed component generation processing.

FIG. 7 is a diagram illustrating a specific example of a boundary-enhanced image concerning a cardiac muscle.

FIG. 8 is a diagram illustrating an internal structure of an image processing unit.

FIG. 9 is a diagram illustrating an internal structure of a summed component generation unit.

FIG. 10 is a diagram illustrating an internal structure of a sample direction DS unit.

FIG. 11 is a diagram illustrating an internal structure of the DS unit.

FIG. 12 is a diagram illustrating an internal structure of a sample direction US unit.

FIG. 13 is a diagram illustrating an internal structure of the US unit

FIG. 14 is a diagram illustrating an internal structure of a summed component calculation unit.

FIG. 15 is a diagram illustrating an internal structure of a multiresolution decomposition unit.

FIG. 16 is a diagram illustrating an internal structure of a boundary component calculation unit.

FIG. 17 is a diagram illustrating a specific example of a fundamental function of non-linear processing.

FIG. 18 is a diagram illustrating a specific example in which the maximum value is varied.

FIG. 19 is a diagram illustrating a specific example in which gain is varied.

FIG. 20 is a diagram illustrating non-linear processing having different properties between a positive value and a negative value.

FIG. 21 is a diagram illustrating a specific example of parameter modification for each level.

FIG. 22 is a diagram illustrating a specific example of weighting processing with reference to a Gₙ component.

FIG. 23 is a diagram illustrating a specific example of weighting processing with reference to a Gₙ component.

FIG. 24 is a diagram illustrating an internal structure of a boundary component add-up unit.

### DESCRIPTION OF EMBODIMENTS

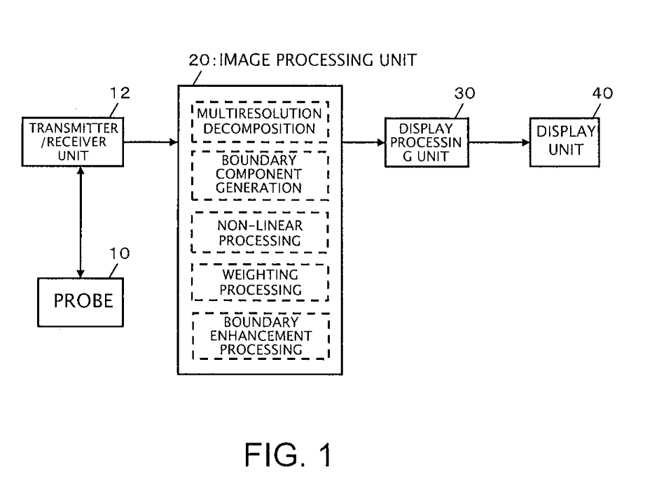

FIG. 1 is a diagram illustrating an overall structure of an ultrasound diagnostic device which is suitable for implementation of the present invention. A probe 10 is an ultrasound probe which transmits and receives ultrasound to and from an area including a subject for diagnosis, such as a heart, for example. The probe 10 includes a plurality of transducer elements, each of which transmits and receives ultrasound, and the plurality of transducer elements are controlled by a transmitter/receiver unit 12 for transmission and reception of ultrasound to form a transmitted beam. The plurality of transducer elements also receive ultrasound from the area including the subject for diagnosis and output signals thus obtained to the transmitter/receiver unit 12. The transmitter/receiver unit 12 then forms a received beam and collects echo data along the received beam. The probe 10 scans an ultrasound beam (the transmitted beam and the received beam) within a two-dimensional plane. Of course, a three-dimensional probe which scans the ultrasound beam three-dimensionally within a three-dimensional space may be used.

When the ultrasound beam is scanned within an area including the subject for diagnosis and the echo data along the ultrasound beam, that is, line data, is collected by the transmitter/receiver unit 12, an image processing unit 20 forms ultrasound image data based on the collected line data. The image processing unit 20 forms image data of a B mode image, for example.

When forming an ultrasound image (image data), the image processing unit 20 enhances the boundaries of a tissue of the heart or the like within the ultrasound image. In order to enhance the boundaries, the image processing unit 20 has functions of multiresolution decomposition, boundary component generation, non-linear processing, weighting processing, and boundary enhancement processing. The image processing unit 20 applies resolution conversion processing to an ultrasound image obtained by the received signal to thereby generate a plurality of resolution images having different resolutions. The image processing unit 20 further applies non-linear processing to a differential image obtained by comparison among the plurality of resolution images to thereby generate a boundary component related to a boundary included in the image. Enhancement processing is then applied to the ultrasound image based on the boundary component which is generated, so that a boundary-enhanced image is generated. The image processing unit 20 then generates a plurality of image data items representing the heart, which is a subject for diagnosis, for a plurality of frames, and outputs the image data items to a display processing unit 30.

The image processing in the image processing unit 20 may be executed after processing including wave detection, logarithmic transformation, and the like, is applied to a signal obtained from the transmitter/receiver unit 12, and may be further followed by coordinate transformation processing executed by a digital scan converter. Of course, the boundary enhancement processing in the image processing unit 20 applied to a signal obtained by the transmitter/receiver unit 12 may be followed by processing including wave detection, logarithmic transformation, and the like, or the coordinate transformation processing executed in the digital scan converter may be followed by the image processing in the image processing unit 20.

The display processing unit 30 applies coordinate transformation processing for transforming the scanning coordinate system of ultrasound to the display coordinate system of an image to the image data obtained by the image processing unit 20, for example, and further adds a graphic image and the like, as necessary, to form a display image including an ultrasound image. The display image formed in the display processing unit 30 is displayed on a display unit 40.

Among the structures (function blocks) shown in FIG. 1, the transmitter/receiver unit 12, the image processing unit 20, and the display processing unit 30 may be implemented by hardware such as a processor, an electronic circuit, and the like, and a device such as a memory may be utilized for the implementation. A preferable specific example of the display unit 40 is a liquid crystal display, for example.

The structures shown in FIG. 1 other than the probe 10 can also be implemented by a computer, for example. More specifically, the structures shown in FIG. 1 other than the probe 10 (only the image processing unit 20, for example) may be implemented using cooperative use of hardware such as a CPU, memory, hard disk, and the like included in a computer, and software (program) which defines the operations of the CPU and the like.

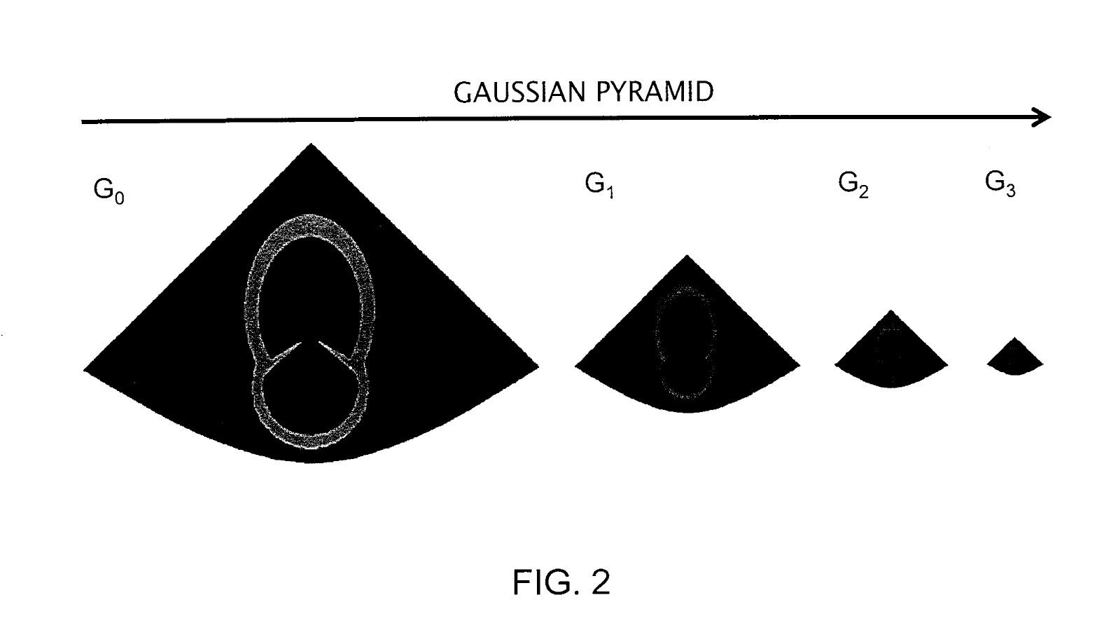

The overall structure of the ultrasound diagnostic device shown in FIG. 1 has been described above. The functions implemented by the ultrasound diagnostic device in FIG. 1 (the present ultrasound diagnostic device) and the like will be described in detailed below. In the following description, the elements (parts) shown in FIG. 1 will be designated by the reference numerals used in FIG. 1. With reference to FIG. 2 to FIG. 7, the principle of the processing executed in the present ultrasound diagnostic device (particularly the image processing unit 20) will be described first. The image processing unit 20 of the present ultrasound diagnostic device enhances boundaries in an ultrasound image using a plurality of resolution images obtained by multiresolution decomposition of the ultrasound image.

FIG. 2 is a diagram illustrating a specific example of multiresolution decomposition, and shows an ultrasound image including cardiac muscle. Specifically, FIG. 2 illustrates an ultrasound image prior to resolution conversion (the original image) G₀, a low-resolution image G₁ obtained through one downsampling processing of the ultrasound image G₀, a low-resolution image G₂ obtained through one downsampling processing of the low-resolution image G₁, and a low-resolution image G₃ obtained through one downsampling processing of the low-resolution G₂.

The image processing unit 20 compares a plurality of resolution images corresponding to different resolutions, e.g. the images G₀ to G₃ shown in FIG. 2, each having different resolutions. Prior to this comparison, upsampling processing is executed in order to make the image size uniform.

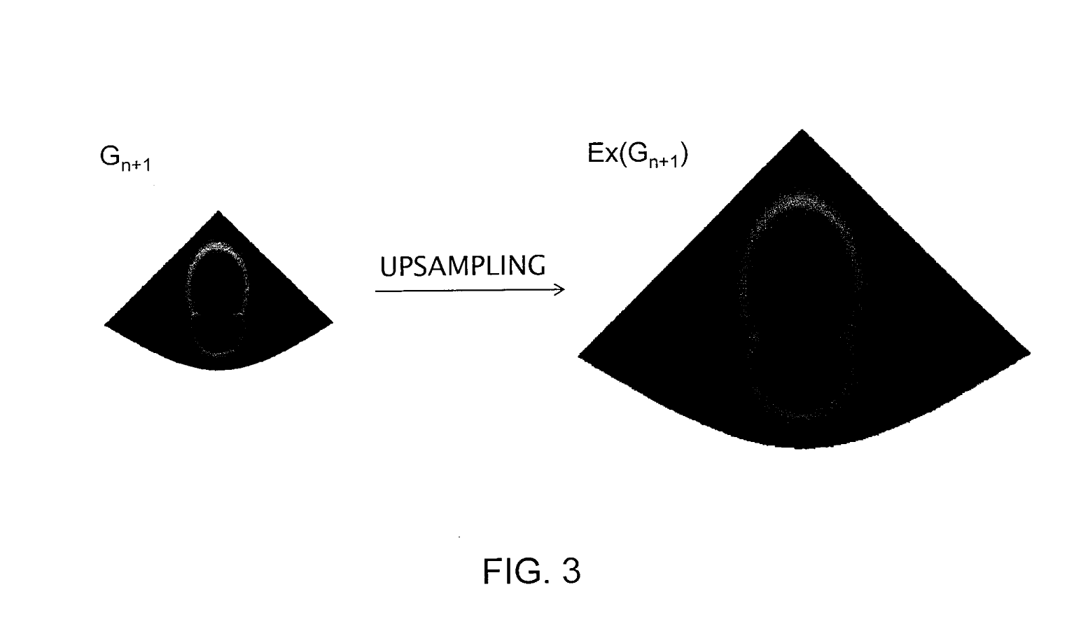

FIG. 3 is a diagram illustrating a specific example of upsampling processing with respect to a resolution image. Specifically, FIG. 3 illustrates a resolution image Ex (Gₙ₊₁) (n is an integer which is 0 or greater) obtained from a resolution image Gₙ₊₁ by one upsampling processing. The resolution image Ex (Gₙ₊₁) has the same resolution as that of the resolution image Gₙ₊₁, and has the same image size as that of the resolution image Gₙ prior to the downsampling processing. The image processing unit 20 generates a differential image based on a plurality of resolution images having different resolutions, e.g. the resolution image Gₙ and the resolution image Ex (Gₙ₊₁).

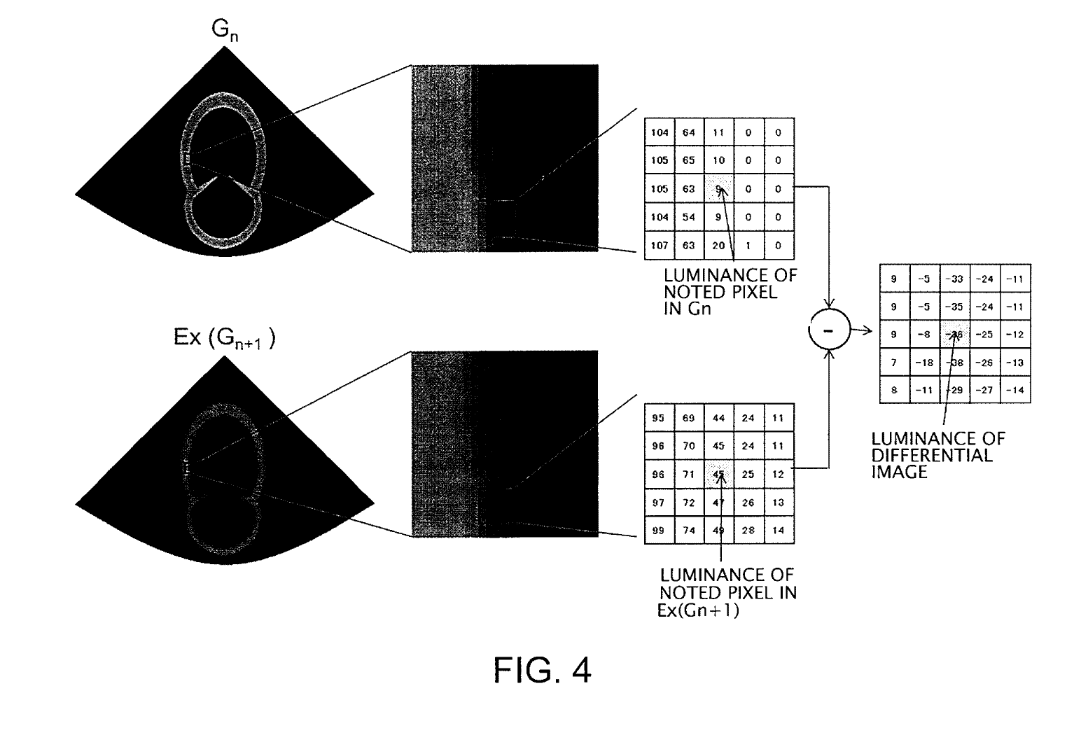

FIG. 4 is a diagram for explaining a differential image. The image processing unit 20 subtracts the resolution image Ex (Gₙ₊₁) from the resolution image Gₙ to form a differential image. More specifically, a difference in the luminance values between corresponding pixels in the two images (pixels at the same coordinates) is defined as a pixel value (a differential luminance value) of the pixel in a differential image.

In an ultrasound image, a cardiac muscle portion of the heart reflects properties of a cardiac muscle tissue (structure), e.g. fine recesses and projections on a tissue surface or within a tissue. Therefore, when a pixel on a cardiac muscle surface or within a cardiac muscle is defined as a pixel of interest, a relatively large difference in luminance appears between the pixel of interest and surrounding pixels in the resolution image Gₙ having a relatively high resolution. A change in the luminance is particularly noticeable at the boundary of the cardiac muscle.

In the resolution image Ex (Gₙ₊₁), which is a dull (blurred) image compared to the ultrasound image Gₙ due to low-resolution processing (downsampling processing), the difference in luminance between the pixel of interest and the surrounding pixels is smaller than that in the ultrasound image Gₙ.

Accordingly, as the difference in luminance between the pixel of interest and the surrounding pixels is greater in the ultrasound image Gₙ, the pixel of interest in the resolution image Ex (Gₙ₊₁) is changed by a greater amount from that in the ultrasound image Gₙ, particularly at the boundary of the cardiac muscle, resulting in a greater pixel value (greater difference in luminance) in a differential image.

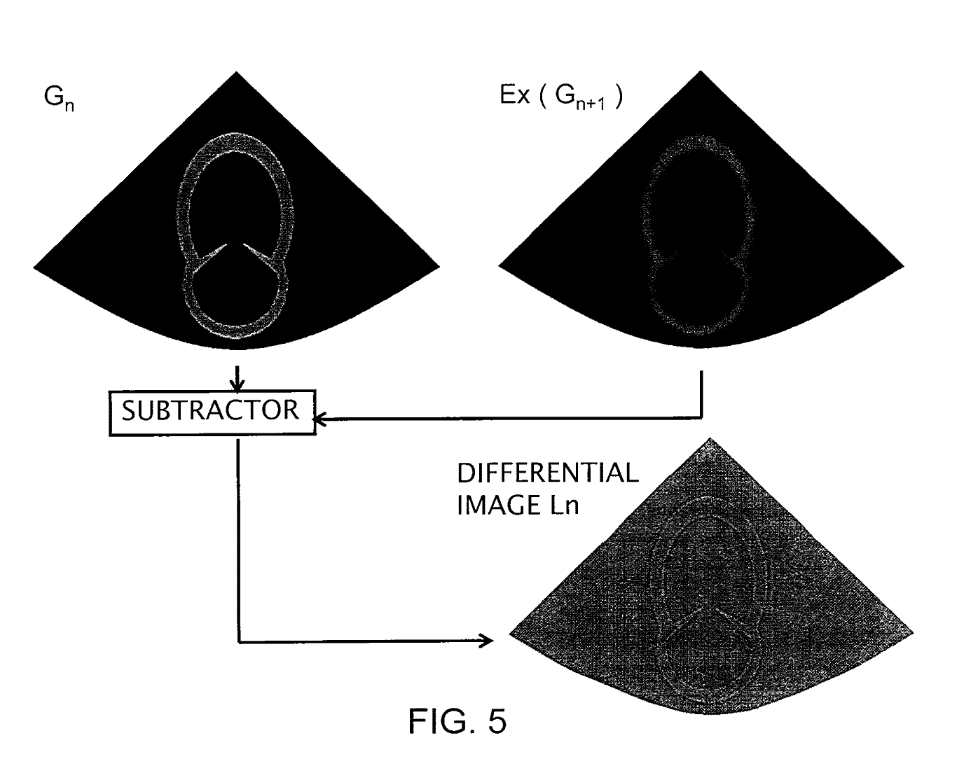

FIG. 5 is a diagram illustrating a specific example of a differential image concerning a cardiac muscle portion. Specifically, FIG. 5 illustrates the resolution image Gₙ (n is an integer which is 0 or greater) and the resolution image Ex (Gₙ₊₁) in a cardiac muscle portion, and a specific example differential image Lₙ between these two images. The image processing unit 20 forms a plurality of differential images from a plurality of resolution images, and, based on the plurality of differential images, generates a summed component for use in enhancing the boundary in an ultrasound image.

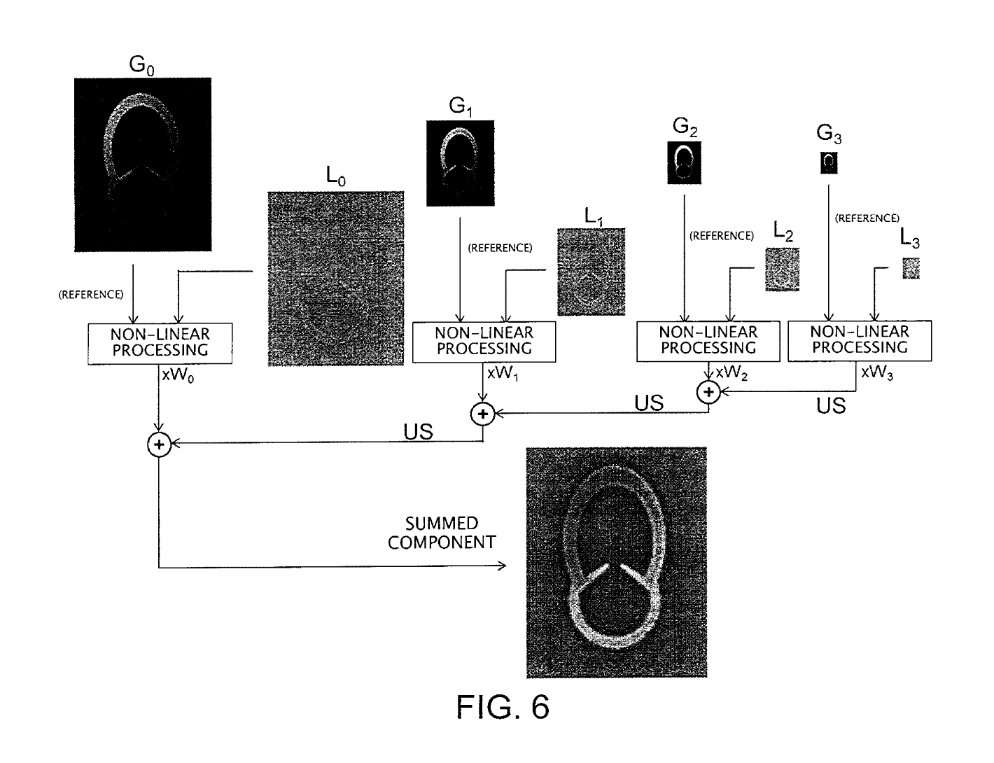

FIG. 6 is a diagram for explaining processing for generating a summed component. The image processing unit 20 generates a summed component based on a plurality of differential images Lₙ (n is an integer which is 0 or greater), for example, based on differential images L₀ to L₃ shown in FIG. 6. A differential image Lₙ is obtained based on a difference between the resolution image Gₙ and the resolution image Ex (Gₙ₊₁) (see FIG. 5).

For generating a summed component, the image processing unit 20 applies non-linear processing to pixels forming each differential image Lₙ. The image processing unit 20 further applies weighting processing with reference to the pixels of the resolution images Gn to the pixels forming each differential image Lₙ which have been subjected to the non-linear processing. The non-linear processing and the weighting processing to be applied to the differential image Lₙ will be described in detail below.

The image processing unit 20 then consecutively sums the plurality of differential images Lₙ having been subjected to the non-linear processing and the weighting processing while applying upsampling (US) processing in a stepwise manner. For the summation, weighting for summation (×Wₙ) may be executed. Thus, the image processing unit 20 generates a summed component based on the plurality of differential images Lₙ.

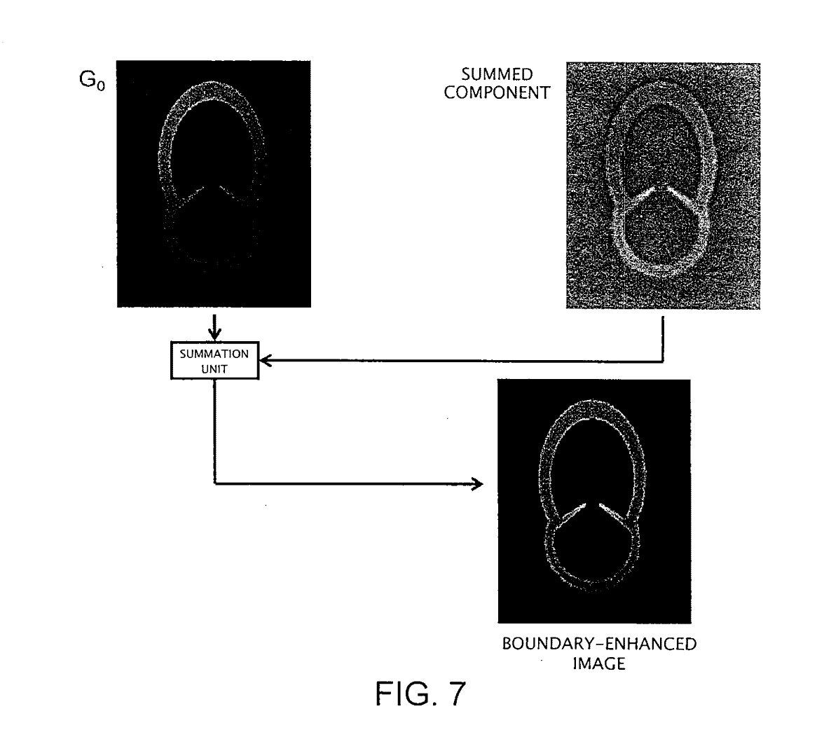

FIG. 7 is a diagram illustrating a specific example boundary-enhanced image concerning a cardiac muscle portion. The image processing unit 20 adds the original image G₀ before the resolution conversion (FIG. 2) and the summed component (FIG. 6), i.e. sums up, for each pixel, the pixel value of the original image and the summed component, thereby forming a boundary-enhanced image having the boundary of the cardiac muscle being enhanced.

The processing which is executed in the present ultrasound diagnostic device (particularly, the image processing unit 20) is summarized as described above. A specific example structure of the image processing unit 20 for implementing the processing described above will now be described.

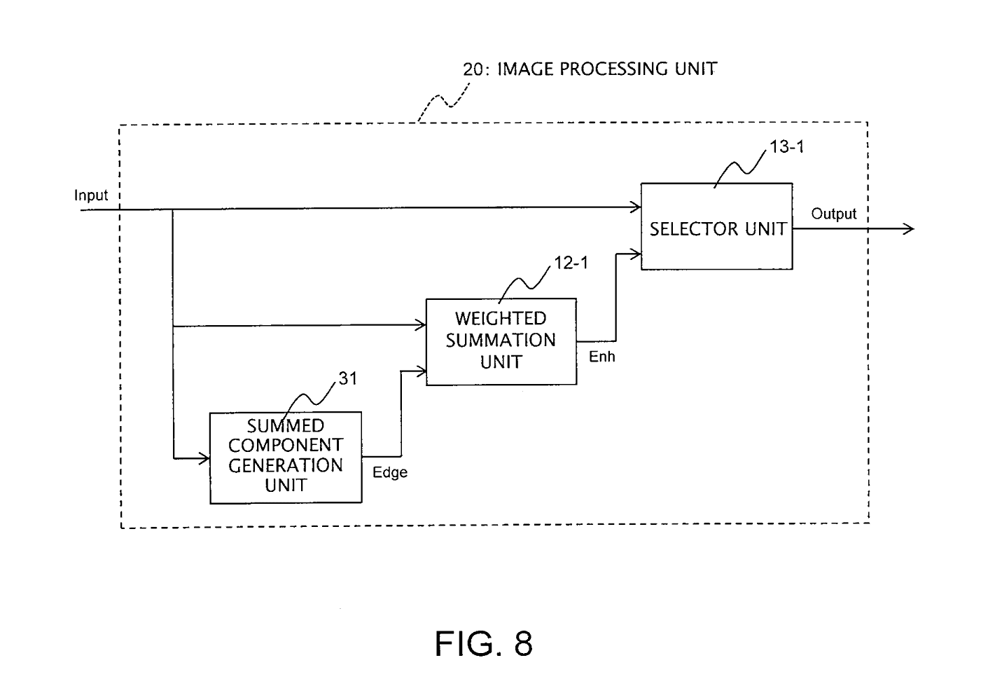

FIG. 8 is a diagram illustrating the internal structure of the image processing unit 20. The image processing unit 20 includes the features as illustrated, and calculates a boundary-enhanced image Enh from an input diagnosis image Input and outputs an image selected by a user on the device from the two images as Output. The diagnosis image Input which is input to the image processing unit 20 is further input to each of a summed component generation unit 31, a weighted summation unit 12-1, and a selector unit 13-1.

The summed component generation unit 31 calculates a summed component Edge through the processing which will be described below. The summed component Edge which is calculated is input to the weighted summation unit 12-1 along with the diagnosis image Input.

The weighted summation unit 12-1 executes weighted summation with respect to the diagnosis image Input and the summed component Edge, to form the boundary-enhanced image Enh. The weighted summation is preferably performed using a parameter W_{org} according to the following equation, but is not limited to this example. The boundary-enhanced image Enh which is calculated is input, along with the diagnosis image Input, to the selector unit 13-1.

Enh=W_{org}·Input+Edge [Mathematical Formula 1]

The selector unit 13-1 receives the diagnosis image Input and the boundary-enhanced image Enh which are input, and performs selection such that the image selected by the user on the device is output as an output image Output. The selected image Output is output to the display processing unit 30.

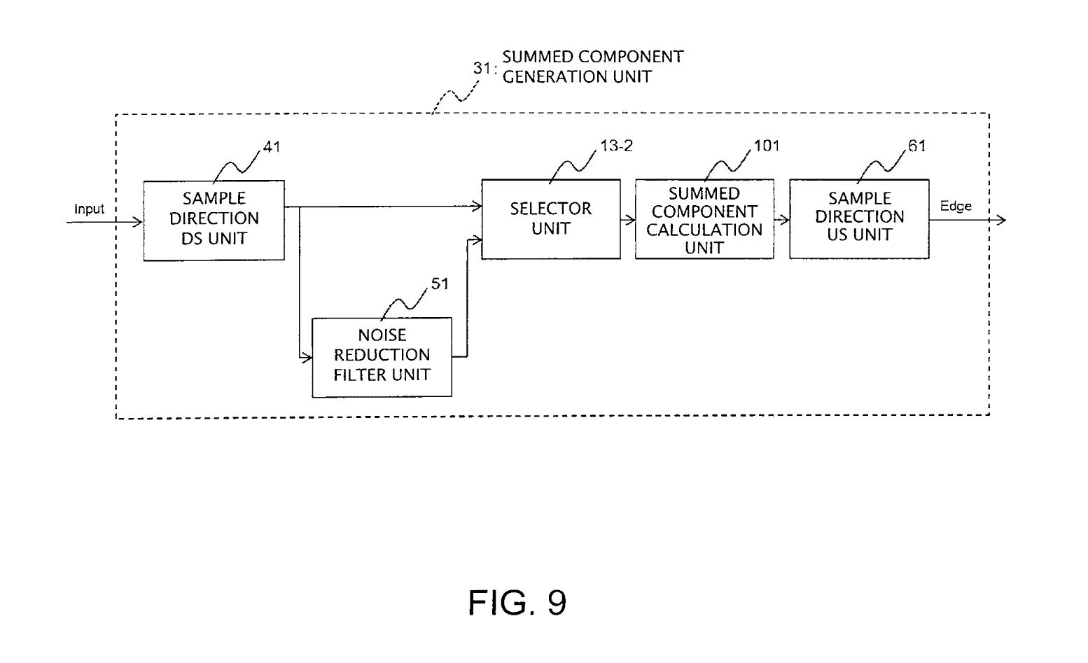

FIG. 9 is a diagram illustrating the internal structure of the summed component generation unit 31 (FIG. 8). The summed component generation unit 31 includes the features as illustrated. The diagnosis image Input which is input to the summed component generation unit 31 is input to a sample direction DS (downsampling) unit 41, where the diagnosis image Input is subjected to downsampling processing in the sample direction (the depth direction of the ultrasound beam, for example) according to the method which will be described below. Data having been subjected to the downsampling processing are then input to a selector unit 13-2 and a noise reduction filter unit 51.

The noise reduction filter unit 51 applies an edge-preserving filter which is called a Guided Filter, for example, to remove noise while preserving boundary information. This structure can reduce noise information to be incorporated in the summed component Edge which is to be calculated through the processing described below. An edge-preserving filter is not limited to the specific example described above, and a non-edge-preserving filter represented by a Gaussian filter or the like may also be used.

The data calculated by the noise reduction filter unit 51 are input, along with the data calculated by the sample direction DS unit 41, to the selector unit 13-2, which outputs data selected by the user on the device to a summed component calculation unit 101.

The summed component calculation unit 101 calculates a boundary image through the processing which will be described below, and inputs the boundary image to a sample direction US (upsampling) unit 61. The sample direction US (upsampling) unit 61 applies upsampling processing to the boundary image in the sample direction according to the method described below to calculate a summed component Edge having the same size as that of the diagnosis image Input which is input to the summed component generation unit 31. The summed component Edge thus calculated is input to the weighted summation unit 12-1 (FIG. 8).

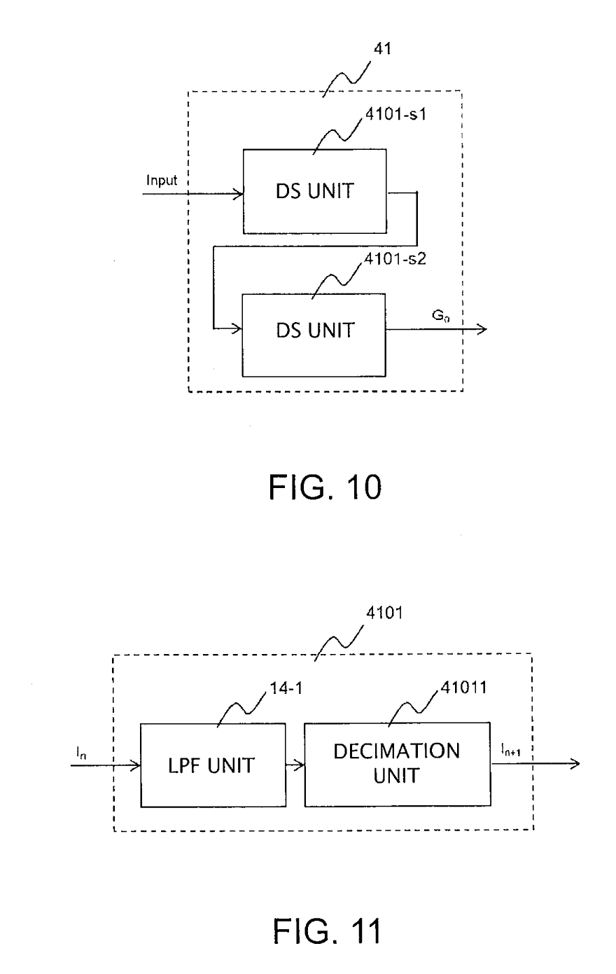

FIG. 10 is a diagram illustrating the internal structure of the sample direction DS unit 41 (FIG. 9). As illustrated, the sample direction DS (downsampling) unit 41 is formed of a plurality of DS (downsampling) units 4101. For clarification of description, in the present embodiment, the sample direction DS unit 41 is formed of two DS units 4101-s1 and 4101-s2, and generates a size-adjusted image G₀ component by downsampling the diagnosis image Input twice. The present invention is not, however, limited to the above specific example. Also, the downsampling in the sample direction may not be performed.

FIG. 11 is a diagram illustrating the internal structure of the DS unit 4101 (FIG. 10). The DS (downsampling) unit 4101 has the features as illustrated. Specifically, an input In component is subjected to low-pass filtering (LPF) by an LPF unit 14-1 and further subjected to decimation processing by a decimation unit 41011, so that an In+1 component having a reduced sample density and a reduced resolution is generated. The DS unit 4101, when performing such processing only in a one dimensional direction, can apply downsampling processing in a one dimensional direction, and the DS unit 4101, when performing such processing in multi-dimensional directions, can execute multi-dimensional downsampling processing.

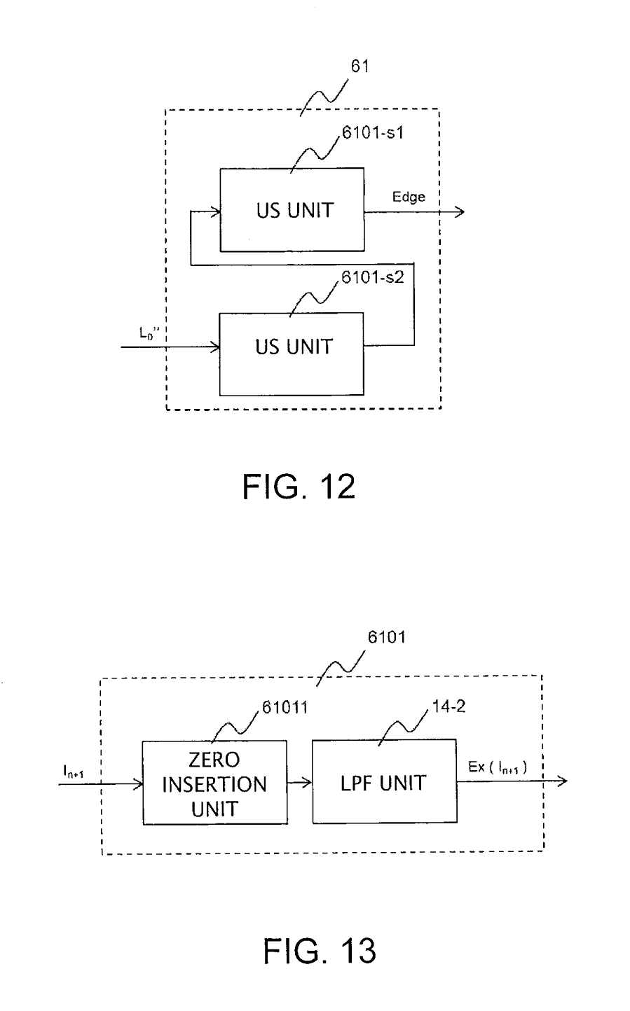

FIG. 12 is a diagram illustrating the internal structure of the sample direction US unit 61 (FIG. 9). As illustrated, the sample direction US (upsampling) unit 61 is formed of a plurality of US (upsampling) units 6101. For clarification of description, in the present embodiment, the sample direction US unit 61 is formed of two US units 6101-s1 and 6101-s2, and generates a summed component Edge by upsampling a boundary image L0″ twice in the sample direction. The present invention is not, however, limited to the above specific example, and it is sufficient that the sample direction US unit 61 outputs a summed component Edge having the same sample density and the same resolution as those of the diagnosis image Input which is input to the summed component generation unit 31 (FIG. 9).

FIG. 13 is a diagram illustrating the internal structure of the US unit 6101 (FIG. 12). The US (upsampling) unit 6101 includes the features as illustrated. Specifically, the input In+1 component is subjected to zero insertion processing in a zero insertion unit 61011 which inserts zero in the input In+1 component at intervals of every other data item, and is further subjected to low-pass filtering (LPF) in an LPF unit 14-2, so that an Ex (In+1) component having an increased sample density is calculated. The US unit 6101, when performing this processing only in a one dimensional direction, can apply upsampling processing in a one dimensional direction, and the US unit 6101, when performing this processing in multi-dimensional directions, can perform upsampling processing in multi-dimensional directions.

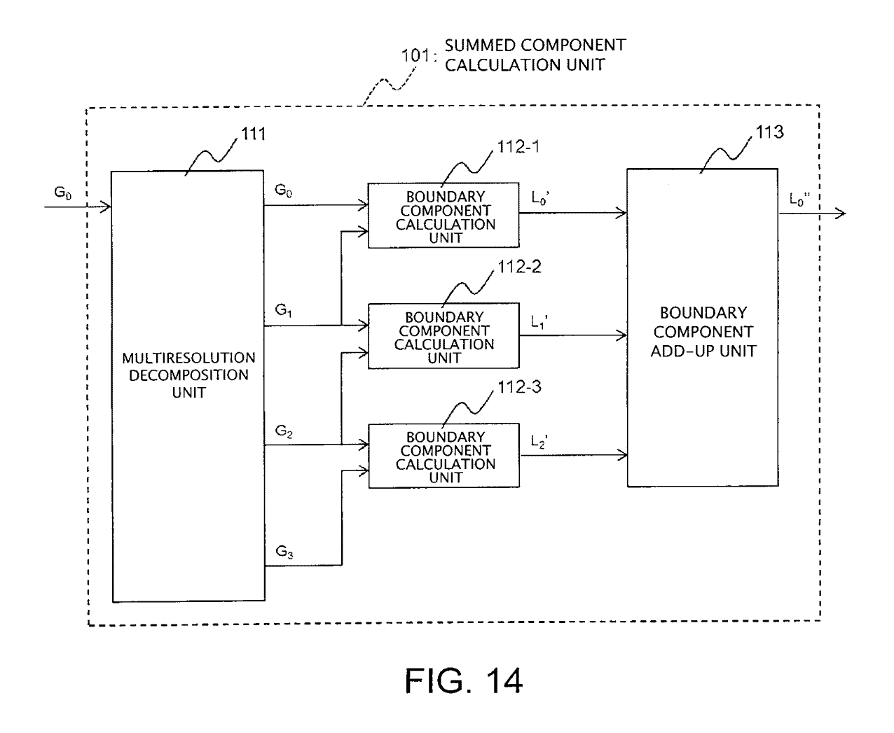

FIG. 14 is a diagram illustrating the internal structure of the summed component calculation unit 101 (FIG. 9). The summed component calculation unit 101 includes the features as illustrated. The input G₀ component which is input to the summed component calculation unit 101 is first input to a multiresolution decomposition unit 111 to undergo multiresolution decomposition through the processing described below. Gₙ components generated by the multiresolution decomposition unit 11 1 are multiresolution representations having sample densities and resolutions that are different from those of the G₀ component.

The Gₙ components calculated in the multiresolution decomposition unit 111 are input, along with Gₙ₊₁ components, to corresponding boundary component calculation units 112-1, 112-2, and 112-3, which calculate Lₙ′ components having been subjected to non-linear processing, through the processing which will be described below. The calculated Lₙ′ components are input to a boundary component add-up unit 113, which generates a boundary image Lₙ″ component through the processing which will be described below.

While in the specific example described above multiresolution decomposition is performed three times, to generate a Gaussian pyramid formed of the Gₙ components (0≦n≦3) and calculate the Lₙ′ components (0≦n≦2), the present invention need not be limited to this example.

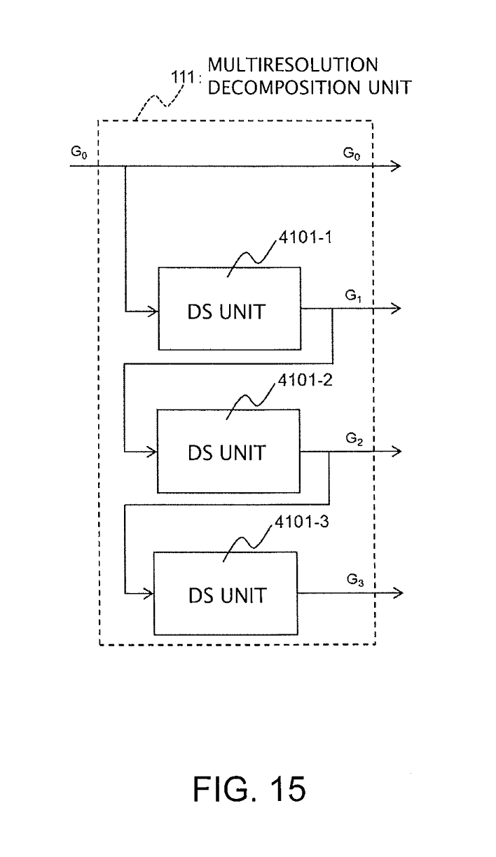

FIG. 15 is a diagram illustrating the internal structure of the multiresolution decomposition unit 111 (FIG. 14). The multiresolution decomposition unit 111 generates a Gaussian pyramid (see FIG. 2) of the input diagnosis image. Specifically, the multiresolution decomposition unit 111 includes the features as illustrated, and the input Gₙ component is input to DS (downsampling) units 4101-1, 4101-2, and 4101-3 to undergo downsampling processing.

While in the above specific example 3 is set to the highest hierarchical level, the present invention is not limited to this example, and multiresolution decomposition may be performed within a scope from level 0 to level n (n≧1). Further, while in the above specific example an example multiresolution decomposition unit is configured to perform Gaussian pyramid processing, the configuration of the multiresolution decomposition unit may be modified to perform multiresolution decomposition using discrete wavelet transform, Gabor transform, bandpass filter in the frequency area, and the like.

The Gₙ component obtained in the multiresolution decomposition unit 111 is further input, along with a Gₙ₊₁ component, to the boundary component calculation unit 112 (FIG. 14).

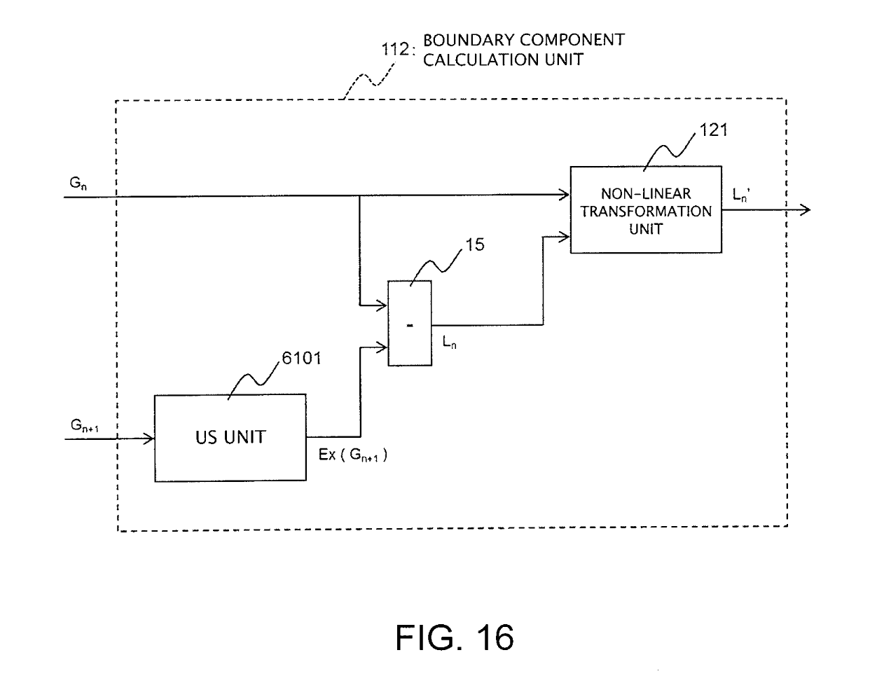

FIG. 16 is a diagram illustrating the internal structure of the boundary component calculation unit 112 (FIG. 14). The boundary component calculation unit 112 includes the features as illustrated. Specifically, the input Gₙ₊₁ component is subjected to upsampling processing in a US (upsampling) unit 6101 to calculate an Ex (Gₙ₊₁) component, which is then input, along with the Gₙ component, to a subtractor 15. The subtractor 15 subtracts the Ex (Gₙ₊₁) component from the Gₙ component, thereby calculating an Lₙ component which is a high frequency component.

In the case of normal Gaussian and Laplacian pyramids, an Lₙ component is output as a high frequency component, and calculation of a summed component using this Lₙ component as an output would result in a summed component Edge including excessive addition and subtraction. Accordingly, in the present embodiment, the Lₙ component is further subjected to non-linear processing in a non-linear transformation unit 121, to calculate an Lₙ′ component.

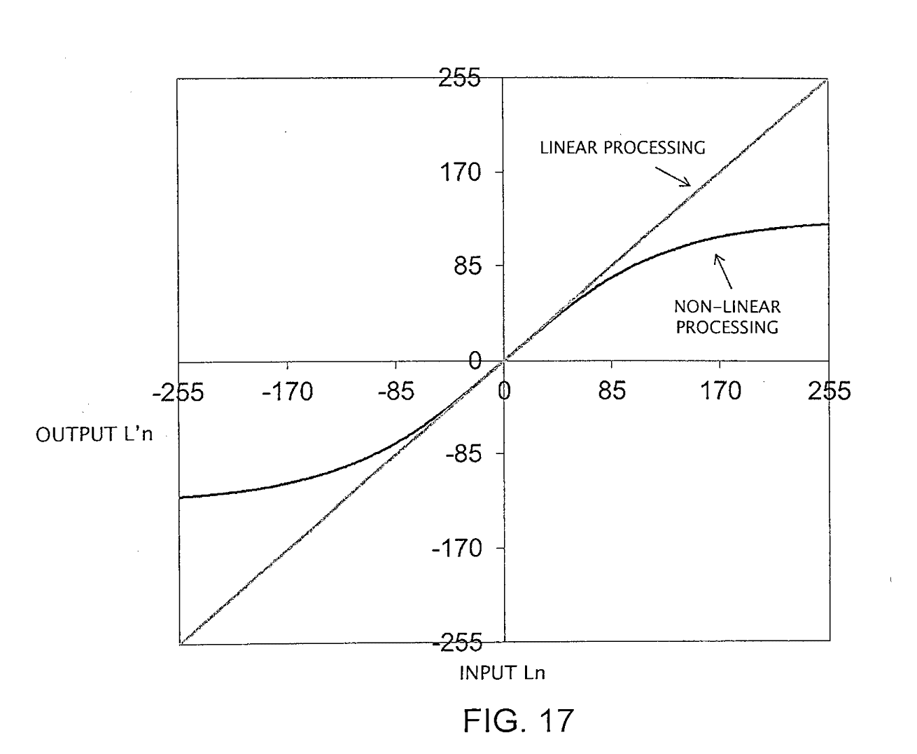

FIG. 17 through FIG. 21 are diagrams illustrating specific examples of non-linear processing. The non-linear transformation unit 121 (FIG. 16) uses a function having linearity near the zero-crossing and having non-linearity appearing further away from the zero-crossing, as represented by a sigmoid function illustrated in FIG. 17 to FIG. 21, for example. The non-linear transform unit 121 configured as described above can obtain an Lₙ′ component which is an output component sufficiently maintaining the boundary component of the Lₙ component, which is an input component, at the zero-crossing with excessive addition and subtraction being suppressed.

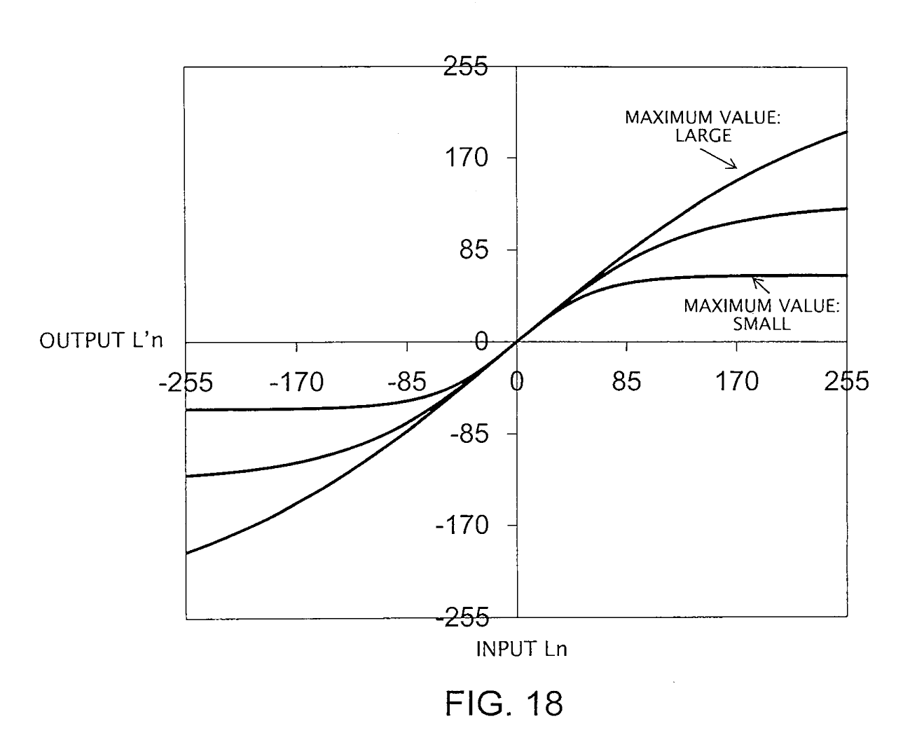

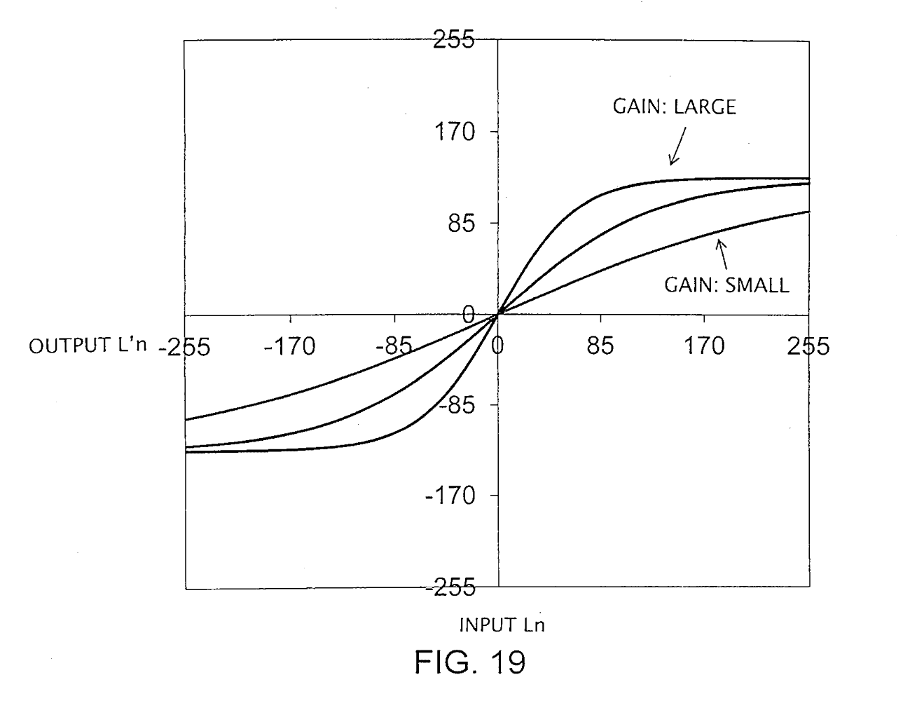

FIG. 17 illustrates a specific example of a basic function of the non-linear processing, FIG. 18 illustrates a specific example in which a parameter related to the magnitude of the maximum value is modified in the basic function of FIG. 17, and FIG. 19 illustrates a specific example in which a parameter related to the magnitude of gain is modified in the basic function of FIG. 17.

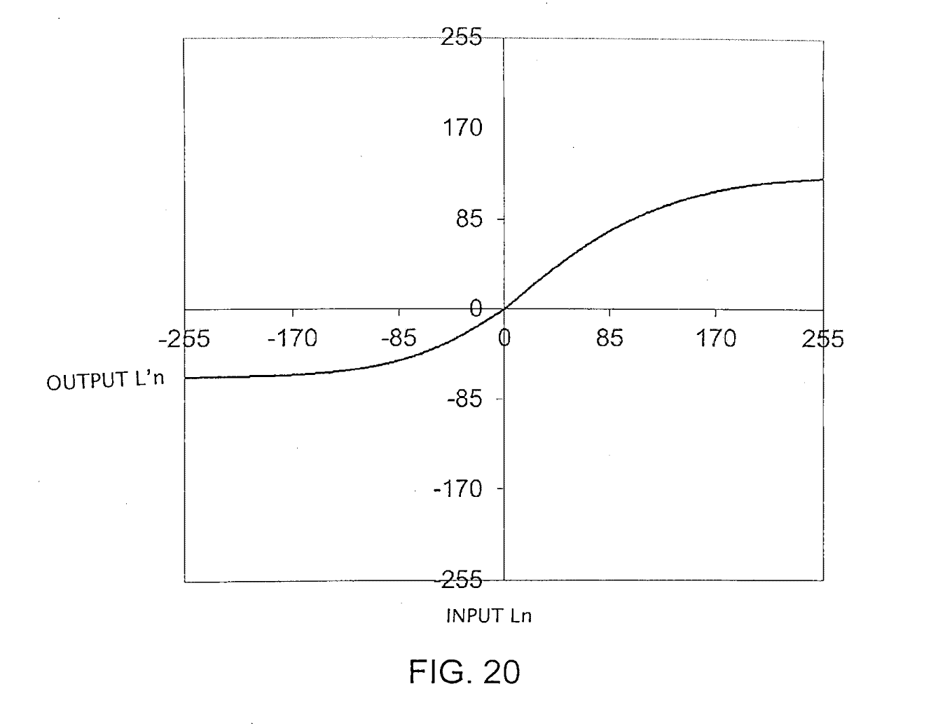

In the present embodiment, the Lₙ component may have either a positive value or a negative value. A negative value as used herein functions to impair information originally contained in the diagnosis image. Accordingly, in order to provide a desirable diagnosis image based on the information inherent in the original diagnosis image, it is desirable that, as illustrated in FIG. 20, a positive value and a negative value are adjusted with different parameters, for example. More specifically, it is desirable to apply non-linear processing having different properties for a positive pixel value and a negative pixel value of the input Lₙ component, particularly non-linear processing with a greater suppression effect for a negative value than for a positive value.

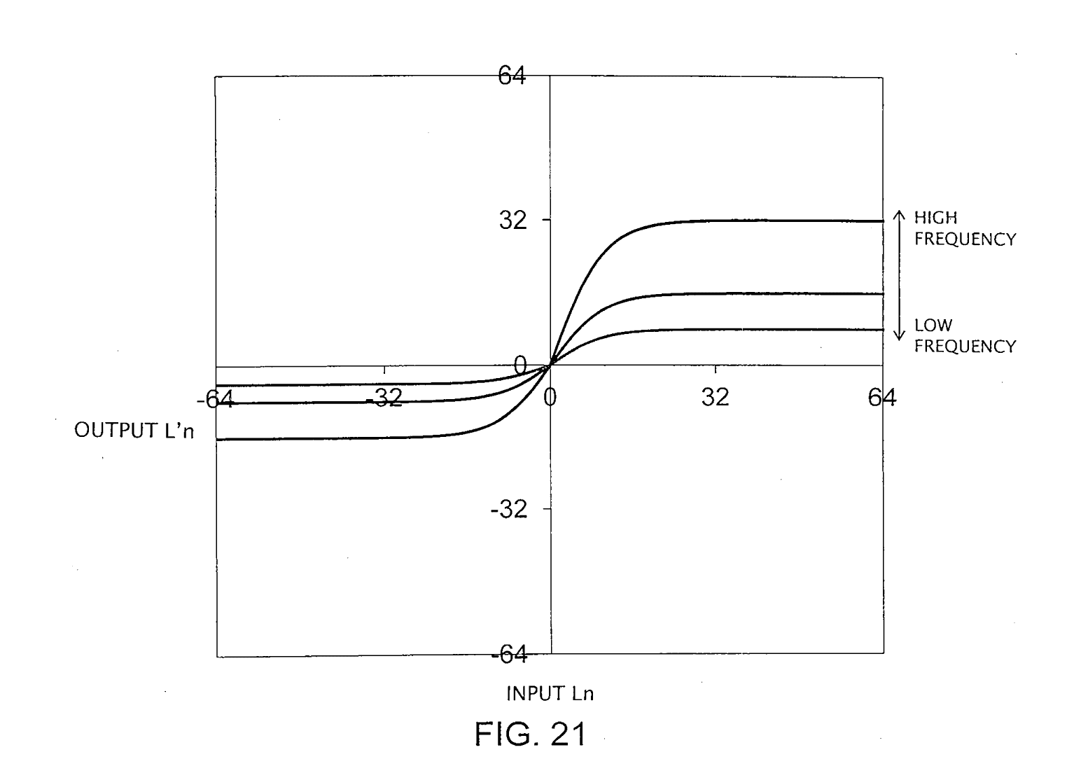

Further, it is preferable to vary the parameters for each level n of the Lₙ component which is a high frequency component in the non-linear processing in the non-linear transformation unit 121 (FIG. 16) of the boundary components calculation unit 112 (FIG. 14), as illustrated in FIG. 21. In order to enhance the high frequency component, for example, the gain or the maximum value near the zero-crossing in the boundary component calculation unit 112-1 is set to a greater value than the gain or the maximum value near the zero-crossing in the boundary component calculation units 112-2 and 112-3. In order to enhance the low frequency component, on the other hand, the gain or the maximum value near the zero-crossing in the boundary component calculation unit 112-3 is set to a greater value than the gain or the maximum value near the zero-crossing in the boundary component calculation units 112-2 and 112-1.

While in the specific example described above it is described that it is preferable to apply non-linear processing in the non-linear transformation unit 121, the present invention is not limited to this example, and a structure may be adopted in which several threshold values are provided and linear transformation is performed for each pair of the threshold values.

As described above, with the non-linear processing applied to the Lₙ component, it is possible to suppress the excessive addition and subtraction, with the boundary component near the zero-crossing being sufficiently maintained. In the present embodiment, in order to reduce the excessive addition and subtraction which causes glare in a posterior wall, for example, which is generated by applying significant addition and subtraction to a portion having a sufficient contrast, such as a high luminance portion, it is further desirable to multiply a component having been subjected to the above-described non-linear processing by a weight determined with reference to the Gₙ component, thereby adjusting the component.

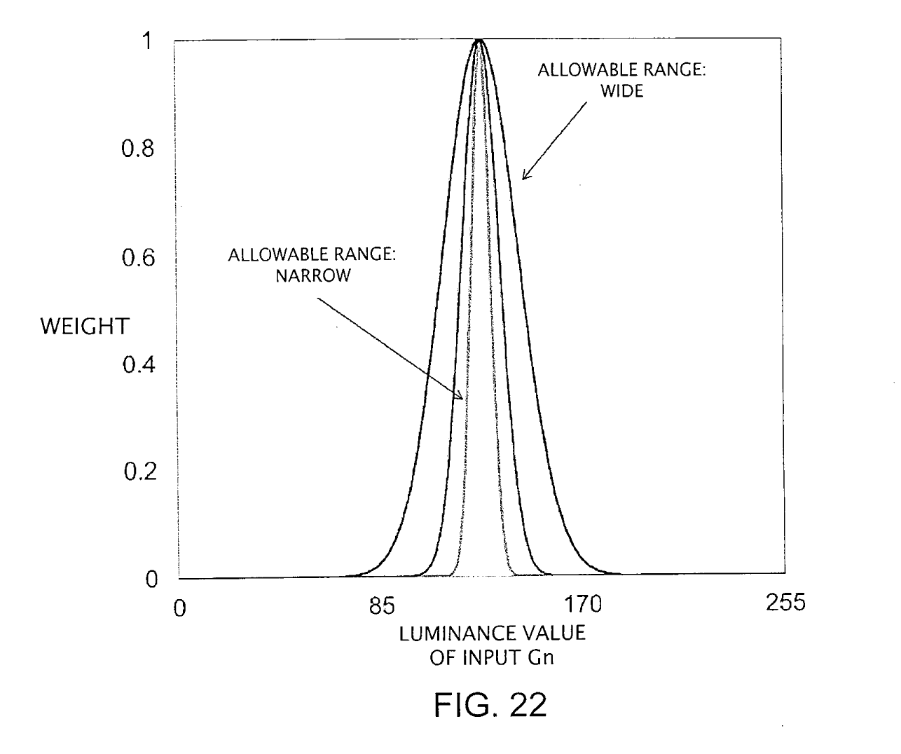

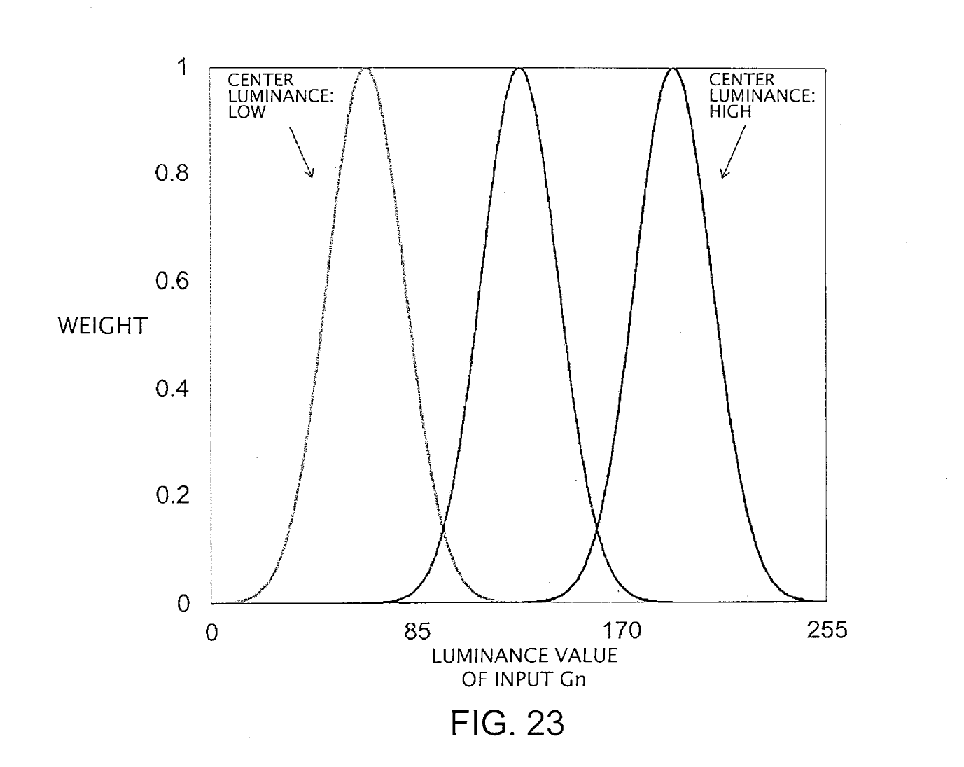

FIG. 22 and FIG. 23 are diagrams illustrating specific examples of weighting processing with reference to the Gₙ component. With the use of the Gaussian functions illustrated in FIGS. 22 and 23, for example, setting the weight to 1 when the pixel of the Gₙ component has a luminance near the edge, and setting the weight toward 0 with respect to a portion with high luminance, such as a posterior wall, or a portion with low luminance, such as the heart cavity, allows suppression of the addition and subtraction with respect to high luminance portions and noise portions.

FIG. 22 shows specific example cases with widened and narrowed parameters related to a range (allowable range) near the edge, and FIG. 23 shows specific example cases with high and low parameters related to the luminance which is judged as an edge (center luminance).

While in the specific example described above a weight to the Lₙ component is determined with reference to the luminance value of the Gₙ component, the present invention is not limited to this example. For example, a weight may be determined with reference to a feature other than the luminance value, such as by setting a weight for a portion with a high edge intensity to 1 and setting a weight for a portion with a low edge intensity to 0, with reference to the boundary intensity.

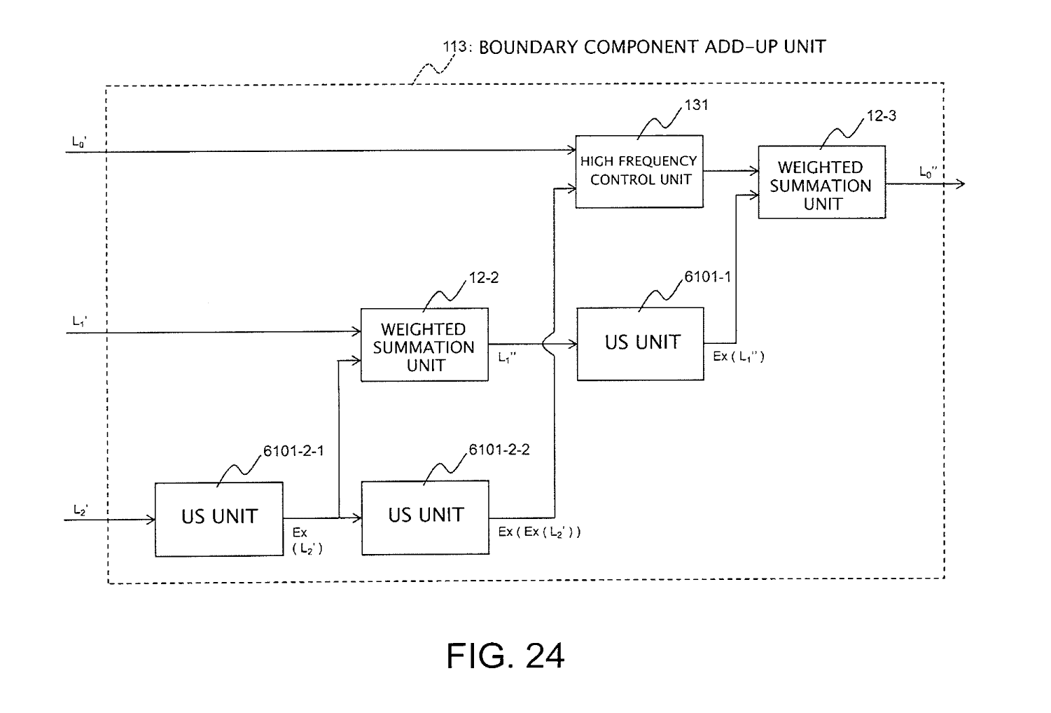

FIG. 24 is a diagram illustrating the internal structure of the boundary component add-up unit 113 (FIG. 14). The boundary component add-up unit 113 has the features as illustrated and generates a boundary image L₀″, based on an L₀′ component, an L₁′ component, and an L₂′ component obtained from the boundary component calculation units 112-1, 112-2, and 112-3 (FIG. 14), respectively. In addition to the L₀′ component, the L₁′ component, and the L₂′ component, more levels may be used.

The L₂′ component which is input is subjected to upsampling in an US (upsampling) unit 6101-2-1, and is then input, as an Ex (L₂′) component, to a weighted summation unit 12-2 and an US (upsampling) unit 6101-2-2.

The weighted summation unit 12-2 applies weighted summation to the L₁′ component and the Ex (L₂′) component to generate an L₁″ component. The weighted summation in the weighted summation unit 12-2 is preferably performed by a calculation using a parameter W₂, according to the following formula, which is not limiting:

$${L}_{1}^{″} = {L}_{1}^{′} + {W}_{2} \cdot Ex ( {L}_{2}^{′} ) \qquad [ Mathematical Formula 2 ]$$

The component calculated in the weighted summation unit 12-2 is further upsampled in an US (upsampling) unit 6101-1, and is input, as an Ex (L₁″) component, to a weighted summation unit 12-3.

The Ex (L₂′) component input to the US unit 6101-2-2 is subjected to further upsampling processing to form an Ex (Ex (L₂′)) component having the same image size as that of the L₀′ component, which is then input to a high frequency control unit 131.

The high frequency control unit 131 removes a noise component from the L₀′ component including a relatively large amount of noise, while leaving the boundary component remaining therein. More specifically, the high frequency control unit 131 calculates weighting such that, when the value of the Ex (Ex (L₂′)) component is large, it is assumed that the component is a component close to the boundary and the weight is set to be close to 1, whereas when the value of the Ex (Ex (L₂′)) component is small, it is assumed that the component is information of a position distant from the boundary of a large structure, and the weight is set toward 0. Further, the weighted value which is calculated is multiplied by the L₀′ component, thereby reducing the noise component included in the L₀′ component. The L₀′ component with the noise component being reduced is input to the weighted summation unit 12-3.

While in the specific example described above the processing for reducing the noise in the L₀′ component with reference to the Ex (Ex (L₂′)) component has been described, the present invention is not limited to this example, and noise reduction processing may be performed with reference to a component having a lower resolution than the Lₙ′ component which is noted, for example.

The weighted summation unit 12-3 performs weighted summation with respect to the L₀′ component having been subjected to noise reduction processing in the high frequency control unit 131 and the Ex (L₁″) component obtained from the US unit 6101-1, to thereby generate the boundary image L₀″. The weighted summation in the weighted summation unit 12-3 is preferably performed by calculation using parameters W₀ and W₁, according to the following formula, which is not limiting:

L″₀=W₀·L′₀+W₁·Ex(L″₁) [Mathematical Formula 3]

The component calculated in the weighted summation unit 12-3 is upsampled in the sample direction US (upsampling) unit 61 (FIG. 9), and is input, as a summed component Edge, to the weighted summation unit 12-1 (FIG. 8).

As described above with reference to FIG. 8, the weighted summation unit 12-1 weighted-sums the diagnosis image Input and the summed component Edge, to form the boundary-enhanced image Enh. The boundary-enhanced image Enh which is calculated is input, along with the diagnosis image Input, to the selector unit 13-1. The selector unit 13-1 performs selection such that an image selected by the user on the device is output as an output image Output. The selected image is then output, as the output image Output, to the display processing unit 30 and displayed on the display unit 40.

In the field of circulatory organs, particularly in the ultrasonography of a heart, for example, evaluation of properties and forms of a tissue is regarded to be significant, and therefore an increase in visibility of the tissue boundaries in the endocardia surface has been desired. Conventional techniques, however, have raised a problem that the boundary enhancement would not only enhance the endocardial surface but also increase the noise in the heart cavity and the glare in the posterior wall, thus producing an image which is not suitable for diagnosis.

The ultrasound diagnostic device according to the present embodiment described above, on the other hand, adds a boundary image which is calculated from an ultrasound image of the examinee and controlled so as not to generate incongruity to the ultrasound image, for example, so that a diagnosis image with the visibility in the tissue boundary increased without incongruity can be generated.

While a preferred embodiment of the present invention has been described, the embodiment described above is only an example and does not limit the scope of the invention. The invention includes various modifications which do not depart from the nature of the invention.

### REFERENCE SIGN LIST

10 probe, 12 transmitter/receiver unit, 20 image processing unit, 30 display processing unit, 40 display unit.

1. An ultrasound diagnostic device, comprising:
a probe configured to transmit and receive ultrasound;
a transmitter/receiver unit configured to control the probe to obtain a received signal of ultrasound;
a resolution processing unit configured to perform resolution conversion processing with respect to an ultrasound image obtained based on the received signal, to thereby generate a plurality of resolution images having different resolutions; and
a boundary component generation unit configured to generate a boundary component related to a boundary included in an image by non-linear processing applied to a differential image obtained by comparing the plurality of resolution images,
wherein a boundary-enhanced image is generated by applying enhancement processing to the ultrasound image based on the boundary component which is obtained.

2. The ultrasound diagnostic device according to claim 1, wherein
the boundary component generation unit performs non-linear processing with different properties for a positive pixel value of the differential image and for a negative pixel value of the differential image.

3. The ultrasound diagnostic device according to claim 1, wherein
the boundary component generation unit performs non-linear processing such that a pixel value of the differential image having a greater absolute value is suppressed by a greater amount before being output.

4. The ultrasound diagnostic device according to claim 2, wherein
the boundary component generation unit performs non-linear processing such that a pixel value of the differential image having a greater absolute value is suppressed by a greater amount before being output.

5. The ultrasound diagnostic device according to claim 1, wherein
the boundary component generation unit applies, to the differential image having been subjected to the non-linear processing, weighting processing in accordance with a pixel value of the resolution image which has been used for comparison for obtaining the differential image, thereby generating the boundary component image.

6. The ultrasound diagnostic device according to claim 2, wherein
the boundary component generation unit applies, to the differential image having been subjected to the non-linear processing, weighting processing in accordance with a pixel value of the resolution image which has been used for comparison for obtaining the differential image, thereby generating the boundary component image.

7. The ultrasound diagnostic device according to claim 3, wherein
the boundary component generation unit applies, to the differential image having been subjected to the non-linear processing, weighting processing in accordance with a pixel value of the resolution image which has been used for comparison for obtaining the differential image, thereby generating the boundary component image.

8. The ultrasound diagnostic device according to claim 1, wherein
the resolution processing unit generates a plurality of resolution images having resolutions which differ from each other in a stepwise manner,
the boundary component generation unit obtains one boundary component based on two resolution images having resolutions which differ from each other by only one step, thereby generating a plurality of boundary components corresponding to a plurality of steps, and
the boundary-enhanced image is generated by applying the enhancement processing to the ultrasound image based on the plurality of boundary components which are generated.

9. The ultrasound diagnostic device according to claim 8, wherein
the boundary component generation unit generates one differential image based on two resolution images having resolutions which differ from each other by only one step, and applies non-linear processing to a plurality of differential images corresponding to a plurality of steps to generate a plurality of boundary components.

10. The ultrasound diagnostic device according to claim 9, wherein
the boundary component generation unit applies non-linear processing with different properties for a positive pixel value of each differential image and for a negative pixel value of each differential image.

11. The ultrasound diagnostic device according to claim 9, wherein
the boundary component generation unit performs non-linear processing such that a pixel value of each differential image having a greater absolute value is suppressed by a greater amount before being output.

12. The ultrasound diagnostic device according to claim 1, wherein
the resolution processing unit forms a plurality of resolution images having a plurality of resolutions which differ from each other in a stepwise manner, and
the boundary component generation unit obtains one boundary component based on two resolution images having resolutions which differ from each other by only one step, thereby generating a plurality of boundary components corresponding to a plurality of steps,
the ultrasound diagnostic device further comprising:
a summed component generation unit configured to generate a summed component of an image based on a plurality of boundary components corresponding to a plurality of steps; and
a summation processing unit configured to add the summed component which is generated to the ultrasound image to thereby generate the boundary-enhanced image.

13. The ultrasound diagnostic device according to claim 12, wherein
the boundary component generation unit generates one differential image based on two resolution images having resolutions which differ from each other by only one step, and applies non-linear processing to a plurality of differential images corresponding to a plurality of steps to generate a plurality of boundary components.
